(19) United States
(12) Patent Application Publication
(10) Pub. No.: US 2006/0275361 A1
(43) Pub. Date: Dec. 7, 2006

(54) Rapidly dissolving gelatin compositions and products made therefrom

(75) Inventors: Hideki Sakanishi, Saitama City (JP); Atul Mistry, Branchburg, NJ (US); Trevour Kelleher, Sparta, NJ (US)

Correspondence Address: HOFFMANN & BARON, LLP, 6900 JERICHO TURNPIKE, SYOSSET, NY, 11791, US

(73) Assignee: Cadbury Adams USA LLC.

(21) Appl. No.: 11/144,331

(22) Filed: Jun. 3, 2005

(51) Int. Cl. A61K 9/64; B29C 39/10

(52) U.S. Cl. 424/456; 264/109; 264/4

(57) ABSTRACT

Rapidly dissolving gelatin compositions and products made therefrom are disclosed. The gelatin compositions and products exhibit controllable dissolution speeds, and particularly faster dissolution times through the incorporation of one or more dissolution enhancing materials.

### FIELD

The present invention generally relates to gelatin compositions comprising gelatin and comestible products made therefrom. The compositions of the invention have enhanced dissolution properties and excellent shelf stability. Capsules and other delivery vehicles made from the gelatin compositions, also demonstrate enhanced dissolution speed such that once placed in the mouth, core material within the capsule or delivery vehicle is also rapidly released.

### BACKGROUND

Gelatin capsules are used to deliver a wide variety of products, ranging from food items to medicine. Typically the gelatin capsule surrounds a core component, such as a solid, gel or a liquid material, which is intended to be released in the body.

Gelatin capsules generally fall into two main classifications: joined capsules; and seamless capsules. Capsules parts which have two shell portions which are designed to fit together, i.e., be joined to define a core area, and which have a seam or junction where the parts are joined, are examples of the first category. Seamless gelatin capsules are conventionally produced by injecting a co-axial stream of the core material and gelatin into a liquid medium, whereupon the coaxial stream breaks up into individual seamless capsules.

A disadvantage common to conventional gelatin capsules, both seamless and otherwise, relates to the relatively slow dissolution time once placed in the mouth of the consumer. Although gelatin capsules are generally water-soluble, they tend to require several minutes in the month, before sufficient dissolution occurs to release their core materials. The incorporation of additives to effectuate faster dissolution has been considered, but this approach often deleteriously affected stability.

Accordingly, there is a need for gelatin capsules which dissolve more quickly in the mouth, and yet have acceptable stability, as well as organoleptic properties.

### BRIEF DESCRIPTION OF THE DRAWINGS

FIG. 1 graphically depicts the average time it took for formulations of the invention and control formulations to dissolve in the mouths of human respondents.

FIG. 2 shows the correlation between the data depicted in FIG. 1 and dissolution data obtained from similar sample formulations using a spectrophotometer.

FIG. 3 demonstrates the hardness of typical formulations of the invention increasing as a function of time.

### SUMMARY

The present invention provides gelatin compositions and products made from such compositions, such as capsules and other delivery vehicles, which have controllable dissolution times through the incorporation of one or more dissolution enhancing materials.

When water-competing components are employed as the dissolution enhancing materials, they may be incorporated into various physical states such as liquid, semi-liquid or solid form. When the gelatin composition is exposed to water, the water-competing components, which desirably are distributed throughout the composition, absorb water and permit water penetration at a rapid rate. The speed of the dissolution of the composition is thus enhanced and may be adjusted to be more rapid by the inclusion of the water-competing components. Additionally, products, such as capsules, made from the gelatin compositions of the present invention, similarly, experience more controllable and desirably more rapid dissolution rates when placed in the mouth, as a result of the incorporation of one or more water-competing components.

When one or more water-insoluble components are employed as the dissolution enhancing material, these components may be in the form of particulates, water-swellable materials or other solid or semi-solid materials. The presence of the components serve to disrupt the gelatin matrix and provide greater susceptibility of the composition to dissolution, thereby changing the speed of the dissolution in a controllable manner.

In certain embodiments of the invention, the water-competing components comprise one or more polyol plasticizer which, when present in relatively high amounts, permit for enhanced speed of dissolution. The speed of dissolution is more rapid as compared to the same gelatin compositions which do not incorporate the high amounts of polyol plasticizers. It has also been discovered that such high amount of polyol plasticizers may be incorporated to achieve rapid dissolution, without compromising stability, which is contrary to conventional thinking. In fact, it has been conventionally believed that amounts greater than about 30% of polyol would deleteriously affect stability to the point of commercial unacceptability.

In other embodiments, the inventive gelatin compositions include both one or more water-competing components, such as polyol plasticizers, and one or more water-insoluble components, such as silicon dioxide.

The present invention provides the incorporation of one or more dissolution enhancing materials in amounts sufficient to control and desirably increase the rapid dissolution when placed in the mouth, yet maintain sufficient shelf stability for a commercially acceptable product. The term “shelf stability” is intended to refer to the ability of the product to resist significant change such that it remains a commercially acceptable product for at least six (6) months and desirably greater than twelve (12) months.

In some embodiments, the dissolution enhancing materials serve to disrupt the continuity of the gelatin composition to permit water ingress to occur more rapidly, with the concomitant result of greater and more effective dissolution of the composition. When applied to comestibles such capsules, candies, lozenges and other oral delivery systems, the capsule shells or comestible matrices desirably more rapidly dissolve, thereby releasing any core materials contained therein, as well as any materials which have been incorporated into the gelatin composition per se which makes up the shells or matrices.

In some embodiments, there is provided a capsule- or comestible-forming composition which includes gelatin and a dissolution enhancing material including at least one water-competing component including at least one polyol plasticizer present in an amount of at least about 60% by weight of the total composition.

In another embodiment of the present invention, there is provided a capsule- or comestible-forming composition which includes a dissolution enhancing material including at least one water-insoluble particulate or dispersant, present in amounts sufficient to modify the dissolution rate thereof.

Also provided is a capsule-forming composition which includes a gelatin matrix and at least one dissolution enhancing component including a material selected from water-competing components, water-insoluble dispersant present in amounts sufficient to increase, modify, control or increase dissolution rate of the gelatin matrix, as compared to the same matrix without said amount of dissolution enhancing component, and combinations thereof.

In yet another embodiment, there is provided a capsule-forming composition, including gelatin and at least one dissolution enhancing component including a least one polyol plasticizer present in an amount of at least about 60% by weight of the total composition, wherein the capsule-forming composition has a dissolution time of about 1 to about 10 minutes at room temperature.

In still another embodiment, there is provided a capsule-forming composition including gelatin and at least one dissolution enhancing component including at least one polyol plasticizer present in an amount of at least about 60% by weight of the total composition, wherein the capsule-forming composition has melting point range of about 30° to about 50° C., and desirably about 32° to about 45° C.

Also provided are capsules which include a gelatin shell, the gelatin shell including gelatin and at least one dissolution enhancing component including at least one polyol plasticizer, wherein the polyol plasticizer is at least 60% by weight of the gelatin shell, or at least one water-insoluble component dispersed therein, or a combination of at least one polyol plasticizer present in amounts of 0-60% and at least one water-insoluble component dispersed therein.

In a further embodiment, there is provided a capsule, including a gelatin shell, wherein the gelatin shell has a shell thickness of about 0.05 to about 0.40 mm, and desirably about 0.05 to about 0.10 mm. For example, when the shell ratio is about 10% and the capsule diameter is about 4.0 mm, the thickness of shell is about 0.051 mm. When the capsule is about 8.0 mm in diameter, the shell thickness is about 0.102 mm.

Yet another embodiment provides a capsule, including a gelatin shell, wherein the gelatin shell has a dissolution time of about 1 to about 10 minutes at room temperature.

In still a further embodiment, there is provided a capsule, including a gelatin shell, wherein the gelatin shell has a melting point range of about 30° to about 50° C.

In another embodiment of the invention, the quick dissolving gelatin capsule as described herein may include in its core composition a flavor and/or breath freshening component, alone or in combination with other actives, such as sweeteners, coloring agents, medicaments, bioactive agents, nutraceuticals, drugs, as well as with carriers or fillers.

The present invention also provides a method for making a gelatin capsule, including the steps of:
a) providing a gelatin composition;
b) admixing the gelatin composition with a dissolution enhancing material including one or more components selected from polyol plasticizers, water insoluble components and combinations thereof, wherein the amount of the dissolution enhancing material by weight of the gelatin composition is sufficient modify, control or desirably increase the dissolution speed of the capsules made therefrom, thereby producing a capsule-forming composition; and
c) forming a gelatin capsule therefrom.

In another embodiment, there is provided a seamless gelatin shell, including a dissolution enhancing material comprising at least one polyol plasticizer present in an amount of at least about 60% by weight of the gelatin shell.

In still another embodiment, there is provided a capsule, including a seamless gelatin shell, the seamless gelatin shell including a dissolution enhancing material including at least one polyol plasticizer present in an amount of at least about 60% by weight of the gelatin shell, wherein the seamless gelatin shell has a shell thickness of about 0.05 to about 0.40 mm.

In a further embodiment, there is provided a capsule, including a seamless gelatin shell, the seamless gelatin shell including a dissolution enhancing material including at least one polyol plasticizer present in an amount of at least about 60% by weight of the gelatin shell, wherein the seamless gelatin shell has a dissolution time of about 1 to about 10 minutes at room temperature.

There is also provided a capsule including a seamless gelatin shell, the seamless gelatin shell including a dissolution enhancing material including at least one polyol plasticizer present in an amount of at least about 60% by weight of the gelatin shell, wherein the seamless gelatin shell has a melting point range of about 30° to about 50° C.

### DETAILED DESCRIPTION

As used herein, the transitional term “comprising,” (and “comprises,” etc.) which is synonymous with “including,” “containing,” and “characterized by,” is inclusive or open-ended, and does not exclude additionally, unrecited elements or method steps, regardless of its use in the preamble or the body of the claim.

As used herein, the term “comestible” is intended to include a wide variety of oral delivery products including capsules (seamless and non-seamless) and confectionery products, such as but not limited to hard candy, soft candy, cotton candy, pressed tablets, chewing gum, film, lozenges, liquid beverages, powdered beverages, and the like.

Capsule-Forming Compositions

Embodiments described herein provide compositions particularly suitable for forming capsules, but other oral delivery vehicles such as comestibles are also contemplated. The capsule-forming compositions of the present invention include gelatin and at least one polyol plasticizer present in an amount of at least about 60% by weight of the total composition.

Gelatins used in the compositions of the present invention may be type A gelatin, type B gelatin, or combinations thereof. Type A gelatin is generally made from pigskin, via an acidic process. Type B gelatin is generally made from cattle hide and bones, via a basic process. Advantageously, the gelatin used may include Type B gelatin.

The gelatin may be present in an amount of about 10% to about 40% by weight of the total composition. Advantageously, the amount of gelatin may be present in amounts of about 20% to about 40% and more desirably about 30% to about 40% by weight of the total composition.

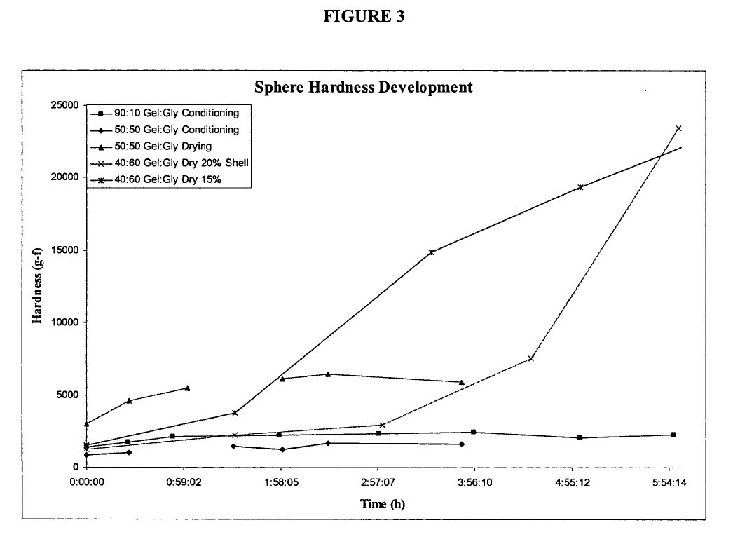

One measurement of a gelatin's consistency is its bloom strength. A “bloom” is a unit assigned to a gelatin composition as a result of the amount of pressure required to push a plunger into a sample of gelatin to a certain depth. In one aspect of the present invention, the gelatin has bloom strength of about 100 to about 300. Depending on how the compositions of the invention are processed, both hard and soft gelatin compositions may be obtained. For example, FIG. 3 shows the hardness of particular compositions of the invention increasing over time. In FIG. 3, various capsules, also referred to as beads, made from the inventive gelatin compositions were tested for their hardness over time. Five capsules were tested. Those indicated by the gelatin: glycerin ratios of 90:10 and 50:50 were comparative compositions. The shells were made either from compositions containing about 90% gelatin and 10% glycerine; or from compositions containing about 50% gelatin and about 50% glycerine. Each of these capsules had a diameter of about 6 mm and shell thicknesses of about 771 cm. In the 90:10 capsule, the capsule also had a shell:liquid centerfill ratio of 90:10. In the 50:50 capsule, the shell:liquid centerfill ratio was also 90:10, i.e., 90% shell and 10% liquid centerfill. Conditioning, where indicated, was performed by washing with ethanol. Drying, where indicated, was performed using air on a rotary pan.

Capsules representative of embodiments of the invention are described as 40:60 gelatin:glycerine dry 20% shell and 40:60 gelatin:glycerine dry 15%. This means the shell is made from about 40% gelatin and 60% glycerine. The diameter of the inventive capsules were also 6 mm. In the inventive capsule indicated as 20% shell, i.e., 20% shell and 80% liquid center, this means that the shell:liquid centerfill ratio was 20:80; and in the capsule indicated as 15% shell, this means that the shell:liquid centerfill ratio was 15:85, i.e., 15% shell and 85% liquid centerfill.

Each of the control and inventive capsules tested for hardness over time using a texture analyzer. The tests were conducted using a projection pin to apply pressure on the surface of the capsule and measure the force applied before it deforms or ruptures. These tests were conducted at about room temperature and the results graphically presented in FIG. 3. All capsules contained flavors, sweetness and color as part of their centerfill.

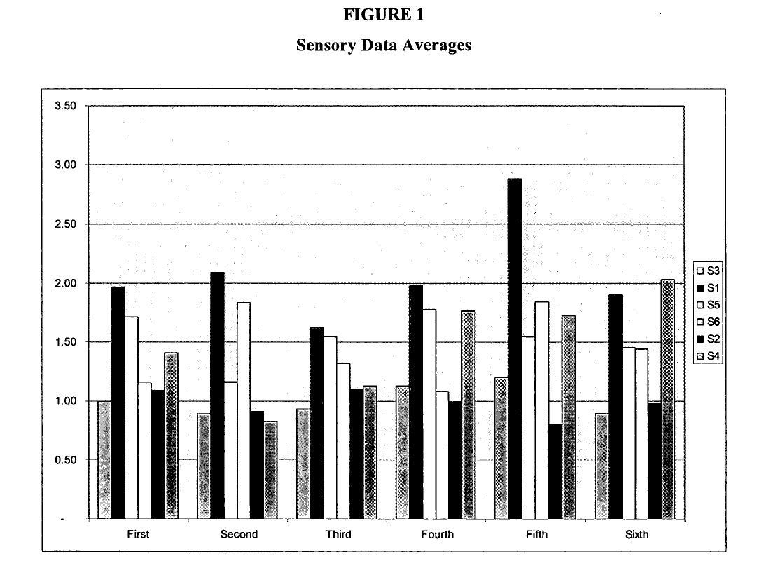

The polyol plasticizer dissolution enhancing component, when used in amounts of at least 60% by weight, allows the composition to dissolve in the consumer's mouth in a relatively short amount of time. As is seen in FIG. 1, the dissolution time of the compositions of the invention is comparable to, or better than that of conventional gelatin formulations. Useful polyol plasticizers include, but are not limited to: glycerin, propylene glycol, sorbitol, maltitol, xylitol, mannitol, erythritol, isomalt, lactitol, and combinations thereof. Advantageously, the polyol plasticizer includes sorbitol, glycerin or a combination thereof.

The polyol plasticizer dissolution enhancing component generally may be present in varying amounts depending on the presence of other components. For example, when used as the sole dissolution enhancing component, the total polyol plasticizer content is desirably at least about 60% or more by weight of the total composition. In some embodiments, the polyol plasticizer is present in amounts from about 60% to about 70% and more desirably about 60% to about 65% by weight of the total composition.

The gelatin compositions may have a dissolution time in water of about 1 to about 10 minutes at room temperature, desirably about 1 to about 8 minutes at room temperature and more desirably about 1 to about 5 minutes at room temperature.

The gelatin compositions may have a melting point range of about 30° to about 60° C., and desirably about 32° to about 45° C. When the gelatin compositions are used as capsule-forming compositions, the capsule shell may have a shell thickness of about 0.05 to about 0.40 mm, and desirably from about 0.05 to about 0.10, as measured by caliper.

The gelatin composition may further include a variety of additional materials, such as fillers, surfactants, active such as those described herein for the core material, and hydrocolloids. Examples of useful hydrocolloids include, but are not limited to, malto dextrin, locust bean gum, gluco mannan, carageenan, pullunan, xanthan gum, carageenan/mannan (50/50), gum arabic, tara gum, agar, pectin high ester pectin, guar gum, gellan gum, cardian, locust bean gum/xanthan (42/58), and combinations thereof.

Examples of suitable water-insoluble dispersant components include, but are not limited to silicon dioxide; metallic stearates, such as magnesium stearate, calcium stearate, and sodium stearate; Carbowax 4000; Carbowax 6000; cornstarch; Cab-O-Sil.

When the dissolution-enhancing material is one or more water-insoluble dispersant components, desirably they are distributed throughout the gelatin composition so as to allow potential chemical and/or physical interactions between the gelatin and the dispersant. The dispersant may modify, control and/or desirably increase the speed of dissolution of the gelatin composition when present in sufficient amounts, by disrupting the spatial arrangement of the individual gelatin strands or molecules. This disruption may prevent efficient cross-linking between individual gelatin molecular strands and is likely to result in a fewer bonding interactions between the strands and increased pathways for water to penetrate, thereby weakening the integrity of the gelatin matrix and allowing for enhanced dissolution rates. Upon exposure to water, such a structurally impaired matrix provides more opportunities for invasion by the water into the matrix, and accordingly, the matrix dissolves more quickly. The water-insoluble particulate components maybe present in amounts of about 0.1% to about 4.0% by weight of the total composition.

Suitable active compositions useful as the core material may be selected from a wide variety of materials including, but not limited to, sweeteners, flavors, medicaments, drugs, cosmetic agents, breath freshening agents, cooling agents, vitamins, coloring agents (colorants, colorings), therapeutic agents, and combinations thereof. For purposes of the present invention, all such materials will be referred to as “actives”.

The amount and type of core material may also vary depending on the particular material chosen and its intended use. Core materials may include hydrophobic components, such as flavor oils or vegetable oils, as well as hydrophilic components. The core compositions may include fillers and other excipients, as well as plasticizers, solvents, surfactants and components used to compound or otherwise prepare the actives for ingestion. The core compositions, as previously mentioned, may take a variety of physical forms, such as powders, granules, gels, pastes or liquids.

In one particular aspect of the invention, a quick dissolving gelatin capsule as described herein may include in its core composition a flavor and/or breath freshening component alone or in combination with other actives, such as sweeteners, coloring agents and carriers or fillers.

Examples of useful drugs (medicaments) include ace-inhibitors, antianginal drugs, anti-arrhythmias, anti-asthmatics, anti-cholesterolemics, analgesics, anesthetics, anti-convulsants, anti-depressants, anti-diabetic agents, anti-diarrhea preparations, antidotes, anti-histamines, anti-hypertensive drugs, anti-inflammatory agents, anti-lipid agents, anti-manics, anti-nauseants, anti-stroke agents, anti-thyroid preparations, anti-tumor drugs, anti-viral agents, acne drugs, alkaloids, amino acid preparations, anti-tussives, anti-uricemic drugs, anti-viral drugs, anabolic preparations, systemic and non-systemic anti-infective agents, anti-neoplastics, anti-parkinsonian agents, anti-rheumatic agents, appetite stimulants, biological response modifiers, blood modifiers, bone metabolism regulators, cardiovascular agents, central nervous system stimulates, cholinesterase inhibitors, contraceptives, decongestants, dietary supplements, dopamine receptor agonists, endometriosis management agents, enzymes, erectile dysfunction therapies such as sildenafil citrate, which is currently marketed as Viagra®, fertility agents, gastrointestinal agents, homeopathic remedies, hormones, hypercalcemia and hypocalcemia management agents, immunomodulators, immunosuppressives, migraine preparations, motion sickness treatments, muscle relaxants, obesity management agents, osteoporosis preparations, oxytocics, parasympatholytics, parasympathomimetics, prostaglandins, psychotherapeutic agents, respiratory agents, sedatives, smoking cessation aids such as bromocryptine or nicotine, sympatholytics, tremor preparations, urinary tract agents, vasodilators, laxatives, antacids, ion exchange resins, anti-pyretics, appetite suppressants, expectorants, anti-anxiety agents, anti-ulcer agents, anti-inflammatory substances, coronary dilators, cerebral dilators, peripheral vasodilators, psycho-tropics, stimulants, anti-hypertensive drugs, vasoconstrictors, migraine treatments, antibiotics, tranquilizers, anti-psychotics, anti-tumor drugs, anti-coagulants, anti-thrombotic drugs, hypnotics, anti-emetics, anti-nauseants, anti-convulsants, neuromuscular drugs, hyper- and hypo-glycemic agents, thyroid and anti-thyroid preparations, diuretics, anti-spasmodics, terine relaxants, anti-obesity drugs, erythropoietic drugs, anti-asthmatics, cough suppressants, mucolytics, DNA and genetic modifying drugs, and combinations thereof.

The flavoring agents which may be used include those flavors known to the skilled artisan, such as natural and artificial flavors. These flavorings may be chosen from synthetic flavor oils and flavoring aromatics and/or oils, oleoresins and extracts derived from plants, leaves, flowers, fruits, and so forth, and combinations thereof. Nonlimiting representative flavor oils include spearmint oil, cinnamon oil, oil of wintergreen (methyl salicylate), peppermint oil, clove oil, bay oil, anise oil, eucalyptus oil, thyme oil, cedar leaf oil, oil of nutmeg, allspice, oil of sage, mace, oil of bitter almonds, and cassia oil. Also useful flavorings are artificial, natural and synthetic fruit flavors such as vanilla, and citrus oils including lemon, orange, lime, grapefruit, and fruit essences including apple, pear, peach, grape, strawberry, raspberry, cherry, plum, pineapple, apricot and so forth. These flavoring agents may be used in liquid or solid form and may be used individually or in admixture. Commonly used flavors include mints such as peppermint, menthol, spearmint, artificial vanilla, cinnamon derivatives, and various fruit flavors, whether employed individually or in admixture. Flavors may also provide breath freshening properties, particularly the mint flavors when used in combination with the cooling agents, described herein below.

Other useful flavorings include aldehydes and esters such as cinnamyl acetate, cinnamaldehyde, citral diethylacetal, dihydrocarvyl acetate, eugenyl formate, p-methylamisol, and so forth may be used. Generally any flavoring or food additive such as those described in Chemicals Used in Food Processing, publication 1274, pages 63-258, by the National Academy of Sciences, may be used. This publication is incorporated herein by reference. This may include natural as well as synthetic flavors.

Further examples of aldehyde flavorings include but are not limited to acetaldehyde (apple), benzaldehyde (cherry, almond), anisic aldehyde (licorice, anise), cinnamic aldehyde (cinnamon), citral, i.e., alpha-citral (lemon, lime), neral, i.e., beta-citral (lemon, lime), decanal (orange, lemon), ethyl vanillin (vanilla, cream), heliotrope, i.e., piperonal (vanilla, cream), vanillin (vanilla, cream), alpha-amyl cinnamaldehyde (spicy fruity flavors), butyraldehyde (butter, cheese), valeraldehyde (butter, cheese), citronellal (modifies, many types), decanal (citrus fruits), aldehyde C-8 (citrus fruits), aldehyde C-9 (citrus fruits), aldehyde C-12 (citrus fruits), 2-ethyl butyraldehyde (berry fruits), hexenal, i.e., trans-2 (berry fruits), tolyl aldehyde (cherry, almond), veratraldehyde (vanilla), 2,6-dimethyl-5-heptenal, i.e., melonal (melon), 2,6-dimethyloctanal (green fruit), and 2-dodecenal (citrus, mandarin), cherry, grape, strawberry shortcake, and mixtures thereof.

In some embodiments, the flavoring agent may be employed in either liquid form and/or dried form. When employed in the latter form, suitable drying means such as spray drying the oil may be used. Alternatively, the flavoring agent may be absorbed onto water soluble materials, such as cellulose, starch, sugar, maltodextrin, gum arabic and so forth or may be encapsulated. The actual techniques for preparing such dried forms are well-known.

In some embodiments, the flavoring agents may be used in many distinct physical forms well-known in the art to provide an initial burst of flavor and/or a prolonged sensation of flavor. Without being limited thereto, such physical forms include free forms, such as spray dried, powdered, beaded forms, encapsulated forms, and mixtures thereof.

The amount of flavoring agent employed herein may be a matter of preference subject to such factors as the type of final composition, the individual flavor, and the strength of flavor desired. Thus, the amount of flavoring may be varied in order to obtain the result desired in the final product and such variations are within the capabilities of those skilled in the art without the need for undue experimentation. Flavoring agents are generally present in amounts from about 0.02% to about 5%, and more specifically from about 0.1% to about 2%, and even more specifically, from about 0.8% to about 1.8%, by weight of the composition.

The sweetening agents used may be selected from a wide range of materials including water-soluble sweeteners, water-soluble artificial sweeteners, water-soluble sweeteners derived from naturally occurring water-soluble sweeteners, dipeptide based sweeteners, and protein based sweeteners, including mixtures thereof. Without being limited to particular sweeteners, representative categories and examples include:

(a) water-soluble sweetening agents such as dihydrochalcones, monellin, steviosides, glycyrrhizin, dihydroflavenol, and sugar alcohols such as sorbitol, mannitol, maltitol, and L-aminodicarboxylic acid aminoalkenoic acid ester amides, such as those disclosed in U.S. Pat. No. 4,619,834, which disclosure is incorporated herein by reference, and mixtures thereof;

(b) water-soluble artificial sweeteners such as soluble saccharin salts, i.e., sodium or calcium saccharin salts, cyclamate salts, the sodium, ammonium or calcium salt of 3,4-dihydro-6-methyl-1,2,3-oxathiazine-4-one-2,2-dioxide, the potassium salt of 3,4-dihydro-6-methyl-1,2,3-oxathiazine-4-one-2,2-dioxide (Acesulfame-K), the free acid form of saccharin, and mixtures thereof;

(c) dipeptide based sweeteners, such as L-aspartic acid derived sweeteners, such as L-aspartyl-L-phenylalanine methyl ester (Aspartame N-[-N-(3,3-dimethylbutyl)-L-α-aspartyl]-L-phenylalinine methyl ester (Neotame)) and materials described in U.S. Pat. No. 3,492,131, L-alphaaspartyl-N-(2,2,4,4-tetramethyl-3-thietanyl)-D-alaninamide hydrate (Alitame), methyl esters of L-aspartyl-L-phenylglycerine and L-aspartyl-L-2,5-dihydrophenyl-glycine, L-aspartyl-2,5-dihydro-L-phenylalanine; L-aspartyl-L-(1-cyclohexen)-alanine, and mixtures thereof;

(d) water-soluble sweeteners derived from naturally occurring water-soluble sweeteners, such as chlorinated derivatives of ordinary sugar (sucrose), e.g., chlorodeoxysugar derivatives such as derivatives of chlorodeoxysucrose or chlorodeoxygalactosucrose, known, for example, under the product designation of Sucralose; examples of chlorodeoxysucrose and chlorodeoxygalactosucrose derivatives include but are not limited to: 1-chloro-1′-deoxysucrose; 4-chloro-4-deoxy-alpha-D-galactopyranosyl-alpha-D-fructofuranoside, or 4-chloro-4-deoxygalactosucrose; 4-chloro-4-deoxy-alpha-D-galactopyranosyl-1-chloro-1-deoxy-beta-D-fructo-furanoside, or 4,1′-dichloro-4,1′-dideoxygalactosucrose; 1′,6′-dichloro 1′,6′-dideoxysucrose; 4-chloro-4-deoxy-alpha-D-galactopyranosyl-1,6-dichloro-1,6-dideoxy-beta-D-fructofuranoside, or 4,1′,6′-trichloro-4,1′,6′-trideoxygalactosucrose; 4,6-dichloro-4,6-dideoxy-alpha-D-galactopyranosyl-6-chloro-6-deoxy-beta-D-fructofuranoside, or 4,6,6′-trichloro-4,6,6′-trideoxygalactosucrose; 6,1′,6′-trichloro-6,1′,6′-trideoxysucrose; 4,6-dichloro-4,6-dideoxy-alpha-D-galacto-pyranosyl-1,6-dichloro-1,6-dideoxy-beta-D-fructofuranoside, or 4,6,1′,6′-tetrachloro4,6,1′,6′-tetradeoxygalacto-sucrose; and 4,6,1′,6′-tetradeoxy-sucrose, and mixtures thereof; and

(e) protein based sweeteners such as thaumaoccous danielli (Thaumatin I and II);

(f) sweeteners such as monatin (2-hydroxy-2-(indol-3-ylmethyl)-4-aminoglutaric acid) and its derivatives.

With respect to cooling agents, a variety of well known cooling agents may be employed. For example, among the useful cooling agents are included menthol, xylitol, menthane, menthone, menthyl acetate, menthyl salicylate, N,2,3-trimethyl-2-isopropyl butanamide (WS-23), N-ethyl-p-menthane-3-carboxamide (WS-3), menthyl succinate, 3,1-menthoxypropane 1,2-diol, among others. These and other suitable cooling agents are further described in the following U.S. patents, all of which are incorporated in their entirety by reference hereto: U.S. Pat. Nos. 4,230,688 and 4,032,661 to Rowsell et al.; U.S. Pat. No. 4,459,425 to Amano et al.; U.S. Pat. No. 4,136,163 to Watson et al.; and U.S. Pat. No. 5,266,592 to Grub et al. These cooling agents may be present in the gelatin shell, in the core composition, or in both. The cooling agents may be present in amounts of about 0.001 to about 10% by weight of the total capsule.

A variety of nutritional supplements may also be included in the compositions of the invention. Virtually any vitamin or mineral may be included. For example, vitamin A, vitamin C, vitamin D, vitamin E, vitamin K, vitamin B₆, vitamin B₁₂, thiamine, riboflavin, biotin, folic acid, niacin, pantothenic acid, sodium, potassium, calcium, magnesium, phosphorus, sulfur, chlorine, iron, copper, iodine, zinc, selenium, manganese, chromium, molybdenum, fluorine, cobalt and combinations thereof, may be used.

Coloring agents may be used in amounts effective to produce a desired color. The coloring agents may include pigments which may be incorporated in amounts up to about 6%, by weight of the composition. For example, titanium dioxide may be incorporated in amounts up to about 2%, and preferably less than about 1%, by weight of the composition. The colorants may also include natural food colors and dyes suitable for food, drug and cosmetic applications. These colorants are known as F.D.& C. dyes and lakes. The materials acceptable for the foregoing uses are preferably water-soluble. Illustrative nonlimiting examples include the indigoid dye known as F.D.& C. Blue No. 2, which is the disodium salt of 5,5-indigotindisulfonic acid. Similarly, the dye known as F.D.& C. Green No. 1 comprises a triphenylmethane dye and is the monosodium salt of 4-[4-(N-ethyl-p-sulfoniumbenzylamino)diphenylmethylene]-[1-(N-ethyl-N-p-sulfoniumbenzyl)-delta-2,5-cyclohexadieneimine]. A full recitation of all F.D.& C. colorants and their corresponding chemical structures may be found in the Kirk-Othmer Encyclopedia of Chemical Technology, 3rd Edition, in volume 5 at pages 857-884, which text is incorporated herein by reference.

Methods of Preparation

The gelatin compositions of the invention are prepared by mixing the components in water at a temperature of about 60° C., stirring for 3 to 10 minutes, and letting the mixture sit for 1 hour.

The present invention is also directed to a process for making a gelatin capsule. In accordance therewith, a gelatin composition is provided. The gelatin composition is admixed with at least one dissolution enhancing material thereby producing a capsule-forming composition. A gelatin capsule is formed therefrom. Gelatin capsules are generally formed by contacting a capsule-forming composition with a capsule mold. Accordingly, in one aspect, formation of the capsule in the capsule-making process may include dipping a capsule mold, i.e., a capsule-shaped member one or more times into a capsule-forming composition, withdrawing the capsule-shaped member and permitting the composition to dry. In another aspect, formation of the capsule in the capsule-making process may include depositing the capsule-forming composition into a capsule mold and applying appropriate heat and/or pressure to form the capsule or capsule part.

The present invention also contemplates a method of making a seamless gelatin capsule. For seamless capsule preparation processes useful in the present invention, see U.S. Pat. No. 5,209,978 to Kosaka et al., U.S. Pat. No. 5,650,232 to Glenn et al., U.S. Pat. No. 5,882,680, to Suzuki et al., and U.S. Pat. No. 6,719,933, to Nakamura et al., the contents of which are incorporated by reference in their entirety. In accordance with the present invention, as a first step, a gelatin composition is provided, the gelatin composition including gelatin and a dissolution enhancing material as disclosed herein. A core composition to be encapsulated is also provided. As a third step, a co-extruded stream of the gelatin composition and core composition is then forced into a liquid medium. The co-extruded stream forms individual seamless capsules of the gelatin composition, which encapsulate the core composition.

### EXAMPLES

Dissolution properties of gelatin compositions of the invention were tested against those of gelatin compositions. Those compositions tested were designated Samples 1 to 6, and the sensory data were evaluated by measuring sample dissolution time in the mouths of six human respondents. Samples 5 and 6 are representative compositions of the invention, while Samples 1 through 4 are various conventional gelatin compositions typically used in gelatin capsules, and here are used as controls. Table 1 shows the gelatin/polyol ratios for each composition.

TABLE 1

| | | Average Time to | |
|---|---|---|---|
| | | Dissolve (Minutes) | |
| | | Spectrophotometer | Sensory |
| Sample | Gelatin/Plasticizer | Data | Data |
| Control | 70:30 (150Bloom gelatin) | 7.9 | 1.01 |
| Sample 1 | | | |
| Control | 90:10 Mane shell | 26.3 | 2.07 |
| Sample 2 | | | |
| Control | 70:30 Current bloom | 19.3 | 1.53 |
| Sample 3 | | | |
| Control | 70:30 not fully dried | 9.7 | 1.44 |
| Sample 4 | | | |
| Inventive | 40:60 (75:25 | 13.9 | 0.98 |
| Sample 5 | Sorbitol:Glycerin) | | |
| Inventive | 40:60 (100% Glycerin) | 12.8 | 1.48 |
| Sample 6 | | | |

Additionally, Table 1 shows the average dissolution time, both as determined by human respondents, and as determined spectrophotometrically. Spectrophotometric data was obtained using a Beckman® Series.500 UV/Vis Spectrophotometer. A 3.000 g bead of the sample is placed into a test cell filled with recirculating liquid at 37° C. to 38° C. The flow rate is set at 0.25 g/second. The liquid may be deionized water or artificial saliva made from deionized water and appropriate amounts of salt. The absorbance wavelength of the sample is checked by running through wavelengths from about 240 nm to about 780 nm. For example, a wavelength of 610 nm could be chosen for blue color beads, and 505 nm could be used for red color beads. The response over the ultraviolet spectrum is tracked over time to ascertain the time necessary for the capsule to dissolve, i.e. when the contents of the capsule are released.

The data in Table 1 show that according to both the spectrophotometer and to the human respondents, compositions of the invention improved dissolution times as compared to the controls.

TABLE 2
Sensory data

| | First | | Second | | Third | | Fourth | | Fifth | | Sixth | |
|---|---|---|---|---|---|---|---|---|---|---|---|---|
| Sample 1 | 1.27 | 0.73 | 0.93 | 0.85 | 1.08 | 0.78 | 1.47 | 0.78 | 1.50 | 0.90 | 0.92 | 0.87 |
| Sample 2 | 2.05 | 1.88 | 2.33 | 1.85 | 1.72 | 1.53 | 1.82 | 2.15 | 3.13 | 2.63 | 2.23 | 1.57 |
| Sample 3 | 1.95 | 1.47 | 0.93 | 1.38 | 1.77 | 1.32 | 2.03 | 1.52 | 1.47 | 1.63 | 1.58 | 1.33 |
| Sample 4 | 1.20 | 1.10 | 2.10 | 1.57 | 1.43 | 1.20 | 1.23 | 0.93 | 1.52 | 2.17 | 1.45 | 1.43 |
| Sample 5 | 1.50 | 0.68 | 0.80 | 1.03 | 1.15 | 1.05 | 1.02 | 0.97 | 0.88 | 0.72 | 0.92 | 1.05 |
| Sample 6 | 1.60 | 1.22 | 0.63 | 1.03 | 0.77 | 1.48 | 1.80 | 1.73 | 1.77 | 1.68 | 2.02 | 2.05 |

Table 2 shows dissolution time of Samples 1 to 6 in each of the of the six respondents' mouths. Each respondent tested each of the Sample formulations twice. Dissolution time is given in minutes. The “Sensory Data” shown in Table 1 presents the averages of the data shown in Table 2.

With reference to the figures, FIG. 1 depicts the average time it took for sample formulations of the invention to dissolve in the mouths of human respondents. Each respondent placed a bead in his or her mouth, and was instructed to move the bead as little as possible once in the mouth. The time was recorded at that moment when the respondent felt the shell disappear completely. Each respondent tested each formulation twice, as shown in Table 2, and FIG. 1 graphically depicts the averages obtained therefrom. The data demonstrate that the formulations of the invention, despite their relatively increased hardness, still dissolve in an acceptable time frame.

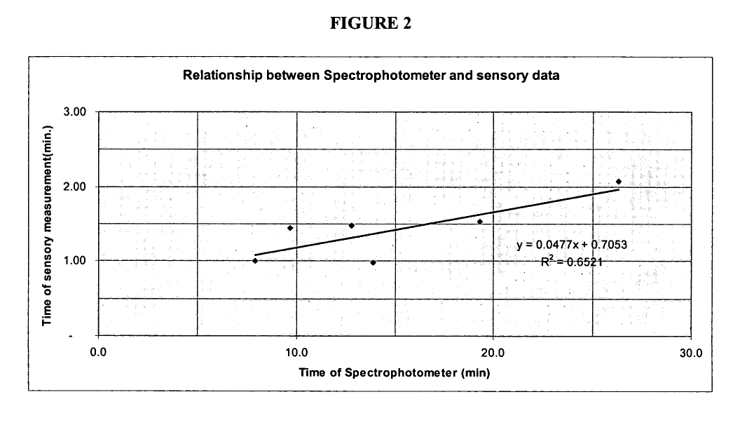

FIG. 2 is a graphic depiction of the correlation between the spectrophotometric data and the sensory data of Table 1. As can be seen, there is a generally linear correlation between the dissolution time as determined by the human subjects, and the dissolution time as determined by the spectrophotometer. This provides some objective evidence as to the accuracy and consistency of the dissolution measurements.

Depending on how the gelatin compositions of the invention are processed, a variety of consistencies can be achieved. For example, FIG. 3 shows two formulations of the invention, each with a ratio of 40:60 gelatin to glycerin, as compared to formulations typically used in gelatin capsules. As can be seen, the hardness of the formulations of the invention increase over time relative to that of the typical formulations, indicating that the gelatinization process was taking place. Nonetheless, as shown in Tables 1 and 2, these hard compositions still maintain organoleptically acceptable dissolution times.

What is claimed is:

1. A capsule-forming composition comprising:
a) gelatin; and
b) at least one dissolution enhancing material selected from the group consisting of at least one water-competing component comprising at least one polyol plasticizer, a water-insoluble particulate component and combinations thereof; wherein the dissolution enhancing component is present in amounts sufficient to control dissolution of the composition.

2. The composition of claim 1, wherein the polyol plasticizer is present in amounts of at least about 60% by weight of the total composition.

3. The composition of claim 1, wherein the polyol plasticizer is present in amounts from about 60% to about 70% by weight of the total composition.

4. The composition of claim 1, wherein the polyol plasticizer is selected from the group consisting of glycerin, propylene glycol, sorbitol, maltitol, xylitol, mannitol, erythritol, isomalt, lactitol, and combinations thereof.

5. The composition of claim 1, wherein the gelatin is present in an amount of about 20% to about 40% by weight of the total composition.

6. The composition of claim 1, wherein the gelatin is selected from the group consisting of type A gelatin, type B gelatin, and combinations thereof.

7. The composition of claim 1, wherein the gelatin has bloom strength of about 100 to about 300 bloom.

8. The composition of claim 1, further comprising a material selected from the group consisting of hydrocolloids, surfactants, actives, fillers and combinations thereof.

9. The composition of claim 5, wherein the hydrocolloid is selected from the group consisting of malto dextrin, locust bean gum, gluco mannan, carageenan, pulluran, xanthan gum, carageenan/mannan (50/50), gum arabic, tara gum, agar, high ester pectin, guar gum resolvent, gellan gum, cardian, locust bean gum/xanthan gum (42/58), and combinations thereof.

10. A composition comprising:
a) a gelatin matrix; and
b) a water-insoluble dispersant present in an amount sufficient to increase the dissolution rate of the gelatin matrix as compared to the same matrix without said amount of dispersant.

11. A composition comprising:
a) gelatin; and
b) at least one polyol plasticizer present in an amount of at least about 60% by weight of the total composition.

12. The composition of claim 11, wherein the capsule-forming composition has a dissolution time of about 1 to about 10 minutes at room temperature.

13. A composition comprising:
a) gelatin; and
b) at least one polyol plasticizer present in an amount of at least about 60% by weight of the total composition,
wherein the composition has melting point range of about 30 to about 50° C.

14. A capsule comprising a gelatin shell,
the gelatin shell comprising gelatin and at least one polyol plasticizer, wherein the polyol plasticizer is at least about 60% by weight of the gelatin shell.

15. A capsule comprising a gelatin shell, wherein the gelatin shell has a shell thickness of about 0.05 to about 0.40 mm.

16. A capsule comprising a gelatin shell, wherein the gelatin shell has a dissolution time of about 1 to about 10 minutes at room temperature.

17. A capsule comprising a gelatin shell, wherein the gelatin shell has a melting point range of about 30 to about 50° C.

18. A process for making a gelatin capsule comprising:
a) providing a gelatin shell composition;
b) admixing the gelatin shell composition with at least one dissolution enhancing material selected from the group consisting of at least one water-competing component, at least one water-soluble particulate component and combinations thereof thereby producing a capsule-forming composition; and
c) forming a gelatin capsule therefrom.

19. The process of claim 18, wherein step c) comprises dipping a capsule mold into the capsule-forming composition.

20. The process of claim 18, wherein step c) comprises depositing the capsule-forming composition into a capsule mold.

21. The process of claim 18, wherein the gelatin capsule is a seamless gelatin capsule.

22. The process of claim 18, wherein the water-competing component comprises at least one polyol in amounts of at least 60% by weight of the total composition.

23. A capsule comprising a seamless gelatin shell,
the gelatin shell comprising a polyol plasticizer present in an amount of at least about 60% by weight of the gelatin shell.

24. A capsule comprising a seamless gelatin shell,
the seamless gelatin shell comprising a polyol plasticizer present in an amount of at least about 60% by weight of the gelatin shell,
wherein the seamless gelatin shell has a shell thickness of about 0.05 to about 0.40 mm.

25. A capsule comprising a seamless gelatin shell,
the seamless gelatin shell comprising a polyol plasticizer present in an amount of at least about 60% by weight of the gelatin shell,
wherein the seamless gelatin shell has a dissolution time of about 1 to about 10 minutes at room temperature.

26. A capsule comprising a seamless gelatin shell,
the seamless gelatin shell comprising a polyol plasticizer present in an amount of at least about 60% by weight of the gelatin shell,
wherein the seamless gelatin shell has a melting point range of about 30 to about 50° C.

27. A method of making a seamless capsule comprising:
a) providing a gelatin composition,
the gelatin composition comprising:
i. gelatin; and
ii. a polyol plasticizer present in an amount of at least 60% by weight of the composition;
b) providing a core composition to be encapsulated; and
c) forcing a co-extruded stream of said gelatin composition and said core composition into a liquid medium whereby the co-extruded stream forms individual seamless capsules of said gelatin composition and encapsulated cores of said core composition.

28. The method of claim 25,
wherein
the core composition is hydrophobic
the gelatin composition is hydrophilic; and
the liquid medium is hydrophobic.
